(19) United States
(12) Patent Application Publication
(10) Pub. No.: US 2022/0304865 A1
(43) Pub. Date: Sep. 29, 2022

(54) EYE MASK

(71) Applicants: National University of Singapore, Singapore (SG); ASPEX OF BIODYNAMICS PTE. LTD, Singapore (SG)

(72) Inventors: Chi Lui HO, Singapore (SG); Ling Rong WONG, Singapore (SG); Hannah Dene JAMES, Singapore (SG)

(21) Appl. No.: 17/607,976

(22) PCT Filed: May 15, 2020
(86) PCT No.: PCT/SG2020/050285

§ 371 Date: Nov. 1, 2021

(30) Foreign Application Priority Data
May 16, 2019 (SG) 10201904424U

(51) Int. Cl. A61F 13/12; B33Y 80/00; A45D 44/00

(52) U.S. Cl. CPC A61F 13/124; B33Y 80/00; A45D 44/002; A61F 13/00063

(57) ABSTRACT

An eye mask for treating and/or preventing periorbital puffiness includes: two side portions extending from a bridge portion, and a retaining component for attaching the eye mask to a face of a user; wherein each side portion includes an orbital region, and an infraorbital region that is configured to apply pressure to a corresponding infraorbital region of the face when the eye mask is attached to the face of the user. Method of manufacturing the eye mask includes obtaining a three-dimensional (3D) facial scan data of the user, generating a mask design, fabricating a mask body by 3D printing and providing, at the mask body, a retaining component.

### TECHNICAL FIELD

The present invention relates, in general terms, to an eye mask for treatment and/or prevention of periorbital puffiness.

### BACKGROUND

Periorbital puffiness under the eyes, which in its milder forms is colloquially referred to as “eye bags”, is common as one ages. The tissues around the eyes, including some of the muscles supporting the eyelids, tend to weaken and the skin around the eyes becomes thinner and may swell or droop. Additionally, the suborbicularis oculi fat pad may increase in size, and together with the aforementioned tissue weakening, may cause the lower eyelids to appear distended. Periorbital puffiness may also occur in other circumstances, such as after surgery.

There have been several proposals for treatment of eye bags. Treatments range from home remedies such as reduction of salt consumption and elevating the head when sleeping, to various creams, gels and patches. However, these treatments suffer from various deficiencies, such as high cost and/or ineffectiveness. Further, none of these known treatments has been found to be effective in prevention of occurrence or re-occurrence of eye bags.

Other possible treatments include the use of medication, such as Furosemide, to reduce and prevent peripheral oedema. However, such treatments are not suitable for general use. Surgical removal of eye bags is also possible, but as for any surgery, inherently carries risks.

It would be desirable to overcome or alleviate at least one of the above-described problems, or at least to provide a useful alternative.

### SUMMARY

Disclosed herein is an eye mask for treating and/or preventing periorbital puffiness, including:
two side portions extending from a bridge portion; and
a retaining component for attaching the eye mask to a face of a user;
wherein each side portion includes an orbital region, and an infraorbital region that is configured to apply pressure to a corresponding infraorbital region of the face when the eye mask is attached to the face of the user.

Also disclosed herein is a kit for treating periorbital puffiness, including a plurality of eye masks according to the preceding paragraph, respective eye masks having respective infraorbital regions, the respective infraorbital regions of respective eye masks being configured to apply progressively greater pressure to the corresponding infraorbital regions of the face.

Further disclosed herein is a method of manufacturing an eye mask for treating and/or preventing periorbital puffiness in a user, including:
obtaining three-dimensional (3D) facial scan data of the user;
generating, from the 3D facial scan data, a mask design, the mask design including two side portions extending from a bridge portion;
wherein each side portion includes an orbital region, and an infraorbital region that is configured to apply pressure to a corresponding infraorbital region of the face of the user when the eye mask is attached to the face;
fabricating a mask body according to the mask design; and
providing, at the mask body, a retaining component for attaching the eye mask to the face of the user.

Further disclosed herein is a method of treating periorbital puffiness in a subject in need thereof, comprising administering at least one eye mask as disclosed herein.

### BRIEF DESCRIPTION OF THE DRAWINGS

Embodiments of the present invention will now be described, by way of non-limiting example, with reference to the drawings in which:

FIG. 1 is a front perspective view of an eye mask according to certain embodiments;

FIG. 2 is a front plan view of the eye mask of FIG. 1;

FIG. 3 is a right side perspective view of an eye mask according to certain embodiments;

FIG. 4 is a schematic cross-sectional view through the line 4-4 of FIG. 2;

FIG. 5 is a schematic cross-sectional view through an alternative eye mask;

FIG. 6 is a schematic cross-sectional view through a further alternative eye mask;

FIG. 7A is a front perspective view of an eye mask according to certain embodiments;

FIG. 7B is a rear perspective view of the eye mask of FIG. 7A;

FIG. 8A is a front plan view of an eye mask according to certain embodiments;

FIG. 8B is a left side perspective view of the eye mask of FIG. 8A;

FIG. 8C is an example of a liner piece of the eye mask of FIG. 8A;

FIG. 8D is an example of a frame piece of the eye mask of FIG. 8A;

FIG. 9A is a front plan view of an eye mask according to further embodiments;

FIG. 9B is a right side view of the eye mask of FIG. 9A;

FIG. 9C is a rear view of the eye mask of FIG. 9A;

FIG. 10 is a flow diagram of a method of manufacturing an eye mask according to certain embodiments; and

FIG. 11 is a schematic depiction of a kit of eye masks when used in treatment of periorbital puffiness.

### DETAILED DESCRIPTION

Embodiments of the present invention generally relate to eye masks and kits for treating and/or preventing periorbital puffiness, to methods of manufacture thereof, and to methods of treatment using such eye masks and/or kits.

The eye mask has two side portions extending from a bridge portion. Each side portion includes an orbital region, and an infraorbital region that is configured to apply pressure to a corresponding infraorbital region of the face when the eye mask is attached to the face of the user. Accordingly, when the eye mask is in place, compression is applied to the areas of the face that are susceptible to puffiness. Advantageously, this targeted compression effect has been found, when the mask is worn during sleep, to reduce puffiness in individuals suffering therefrom. It has also been found to be effective in preventing the occurrence of puffiness. The advantageous effect of the invention can be achieved in as little as a single overnight use for prevention of eye bags.

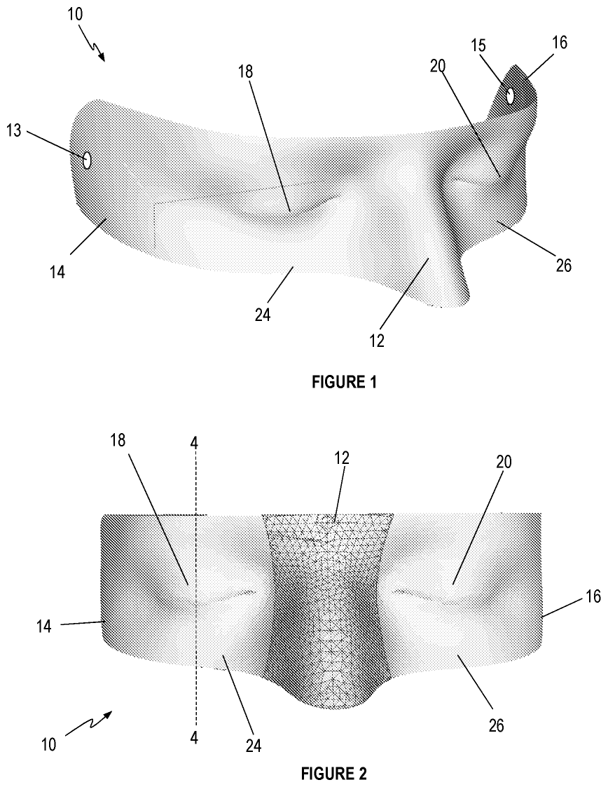

FIGS. 1 and 2 show an example of an eye mask 10 according to certain embodiments. The eye mask 10 includes a mask body including a bridge portion 12 from which extends, on either side, a first side portion 14 and a second side portion 16. First side portion 14 may include an aperture 13 near an extremity thereof, and second side portion 16 may include an aperture 15 near an extremity thereof. Apertures 13, 15 may be used to attach a retaining component, such as a band or strap (not shown), for securing the eye mask 10 to a face of a user. Alternatively, the band may be attached to side portions 14, 16 in other ways, such as by staples, adhesives, or heat-sealing. The band is preferably elastic, and adjustable to accommodate different head shapes while maintaining the compression effect. Advantageously, an elastic band achieves a good compression effect while still ensuring comfort

The side portions 14, 16 in FIGS. 1 and 2 can be integral with the bridge portion 12 such that the mask 10 is formed as a single piece. It will be appreciated, though, that side portions 14, 16 may instead be rigidly coupled to the bridge portion 12.

First side portion 14 includes an orbital region 18 adapted to cover the right eyeball of the user, and an infraorbital region 24 (below orbital region 18) that is configured to apply pressure to a corresponding infraorbital region of the face when the eye mask 10 is attached to the face of the user. Similarly, second side portion 16 includes an orbital region 20 adapted to cover the left eyeball of the user, and an infraorbital region 26 that is configured to apply pressure to a corresponding infraorbital region of the face (also referred to herein as the eye bag region) when the eye mask 10 is attached to the face of the user.

The side portions 14, 16 can have a thinner cross-section compared to the bridge portion 12. In this regard, the orbital regions 18, 20 can have a thinner cross-section compared to the bridge portion 12. In at least some embodiments, the orbital regions and the infraorbital regions can have different thicknesses. For example, the orbital regions 18, 20 of the side portions 14, 16 can have a thinner cross-section compared to the infraorbital regions 24, 26. By forming these portions of the eye mask with a thinner cross-section, they can be made to have a lower rigidity. This is further advantageous as it reduces the pressure on the eye of the subject, and accordingly provides greater comfort to the user.

In some embodiments, the infraorbital regions are made to be thicker than the orbital regions. This increases the rigidity under the eye area for better compression.

The retaining component may be adjustable to vary the pressure applied by the infraorbital regions 24, 26 of the eye mask 10. For example, the retaining component may be a strap having first and second portions that are secured to each other by a fastener such as a hook-and-loop fastener, where the first portion carries hooks (or loops) and is attached to one side portion 14 of the eye mask 10 and the second portion carries loops (or hooks) and is attached to the other side portion 16. The components of the hook and loop fastener may both be carried on one portion that is looped through the other portion and back on itself, for example. In some embodiments, a strap may be formed as a single component having a hook portion on one surface and a loop portion on the opposite surface, wherein the strap is connected to the first side portion 14 and looped back on itself through part of the second side portion 16 (for example, through an aperture 15) to engage the hook portion with the loop portion. The hook portion and loop portion may extend along a substantial part, or the entire length, of the strap to enable greater adjustability.

The retaining component is appropriately sized to provide sufficient tension for holding the eye mask in position. In some embodiments, the tension in the retaining component may be adjustable, for example by way of one or more straps or the like as mentioned above. The retaining component keeps the eye mask in place, for example during restless sleep, without discomfort while the user is lying on their side.

The infraorbital regions 24 and 26 of the eye mask 10 are configured according to the user's face shape. For example, the user's face shape may be determined by a three-dimensional (3D) scan, such as by using a Face Camera Pro of Bellus 3D, Inc. The 3D scan may be performed at the end of the day, when the periorbital region tends to be less swollen. The 3D facial scan data may be analysed to determine the outer limits of the eye ball, and subsequently to generate a mask design that substantially matches the contours of the user's face, particularly in the infraorbital regions, such that when the mask 10 is worn and retained in place with the infraorbital regions 24, 26 contacting the corresponding infraorbital regions of the user's face, the infraorbital regions 24, 26 apply gentle pressure in the areas where swelling tends to occur. Accordingly, wearing the mask 10 overnight produces a compression effect that tends to reduce periorbital puffiness.

As shown in the schematic cross-sectional view of FIG. 4, the eye mask 10 may have an internal (i.e., face-contacting) surface 30 that has a relatively flat profile in the infraorbital region 24. In this version, the eye mask 10 may be particularly suitable for prevention of eye bags.

In some embodiments, such as that shown in FIG. 5, the eye mask 10 may be configured to be suitable for treatment of existing eye bags. A facial scan of the user may be obtained to determine the contours in the infraorbital region. An eye mask 10 may be produced with an infraorbital shape that accommodates the determined contours, but is designed to apply compression to the eye bag. Accordingly, in FIG. 5, the infraorbital region 44 is outwardly recessed or indented (i.e., in a direction away from the user's face) so as to include one or more convex protrusions corresponding to the existing eye bag. The convex protrusion(s) at the infraorbital region 44 allows pressure to be applied to a corresponding infraorbital region of the face when a user suffers from periorbital puffiness, for example in the morning, following surgery or due to an underlying medical condition. The convex shape accommodates the pre-existing eye bag with the inner surface of infraorbital region 44 contacting the eye bag so as to apply gentle compression to it when the eye mask is attached to the face of the user. Once the eye bag has decreased in size, a mask with a protrusion which is less convex (i.e., flatter) may be used, and ultimately, a mask with no protrusion (as shown in FIG. 4) may be used to achieve a smooth extension of the existing cheek line to the eye area. An example of a series of such masks that is used to progressively flatten eye bags is shown in FIG. 11 and described in further detail below.

By progressively flattening the protrusions, an additional targeted compression effect can be achieved in the infraorbital region, without the user needing to tighten the mask. Accordingly, the compression effect can be enhanced without increasing discomfort to the user. The indented or recessed infraorbital region 44 may have a depth between about 3 mm and 20 mm relative to the infraorbital region 24, such that varying degrees of compression may be applied, with smaller depth producing greater compression.

In certain embodiments, each infraorbital region 24, 26 of the eye mask includes an elongate compression region, for example a substantially crescent-shaped region. An elongate compression region provides greater contact surface area, and thus more consistent pressure during use.

The bridge portion 12 is shaped such that it substantially conforms to the contours of a nasal region of a subject. The bridge portion 12 can cover a substantial area of the nasal region, or at least a portion of the nasal region. For example, the b ridge portion can be a nose clip. The bridge portion 12 may be more rigid than the side portions 14, 16. This helps to reinforce the compression effect on the infraorbital regions when the user wears the mask 10. For example, the bridge portion 12 may be made from a more rigid material than the side portions 14 and 16. Alternatively, the bridge portion 12 may be made of the same material as the side portions 14, 16, but may have a greater thickness than the side portions 14, 16. Where the mask 10 is formed by 3D printing, this may be achieved by, for example, printing additional layers of material in the bridge portion 12.

Alternatively, the bridge portion can be thickened such that it can provide structural balance and support. The bridge portion can have a thickness of about 1 mm to about 10 mm, about 1 mm to about 8 mm, about 1 mm to about 6 mm, or about 1 mm to about 4 mm.

The bridge portion 12 can further provide a compression force acting on both sides of a dorsum nasi of the nasal region. This provides a further advantage of keeping the eye mask in position when in use. To this end, when manufacturing the eye mask, the bridge portion can be fabricated to be slightly narrower than the nasal region of a subject.

The eye mask 10 (as well as other embodiments of eye masks described below) may be used in different applications.

In one application, the eye mask 10 may be used as a “preventative” mask for users suffering from early-morning eye swelling. A preventative mask has a shape conforming to the user's normal face shape during the day, to create a targeted compression effect to prevent fluid from leaking into the subcutaneous tissue at night time. For example, the preventative mask aims to keep the face shape in comformity with that determined from an original face scan of the user corresponding to the user's “normal” face shape.

In another application, a “treatment” mask or set of masks can be provided for users with existing (usually familial) eye bags. A first mask targets compression based on the initial shape of the eye bags, and subsequent masks may gently increases pressure by providing successively less convex (flatter) infraorbital regions, which gradually and progressively reduce the swelling. An example of a kit containing treatment masks is depicted schematically in FIG. 11. A first mask 10.1 of a series of n masks has convex protrusions in the infraorbital regions 24.1 and 26.1. A subsequent mask 10.2 has flatter convex protrusions than those of mask 10.1, in infraorbital regions 24.2 and 26.2. Finally, an n^{th} mask 10.n does not have convex protrusions in the infraorbital regions 24.n and 26.n. As depicted in FIG. 11, n=3, but a smaller or larger number of masks could be used as part of a treatment kit. The total number of masks in a mask set is typically dependent upon the size of the existing eye bag.

The eye mask 10 (or any other eye masks disclosed herein) may be configured for treatment of periorbital puffiness by being shaped in the infraorbital regions to produce targeted pressure in a generally upward direction (e.g. from below the infraorbital region towards the infraorbital or orbital region), or straight on (horizontally), or from the edges of the eyebag inwards (towards the nose), or a combination of two or more of these.

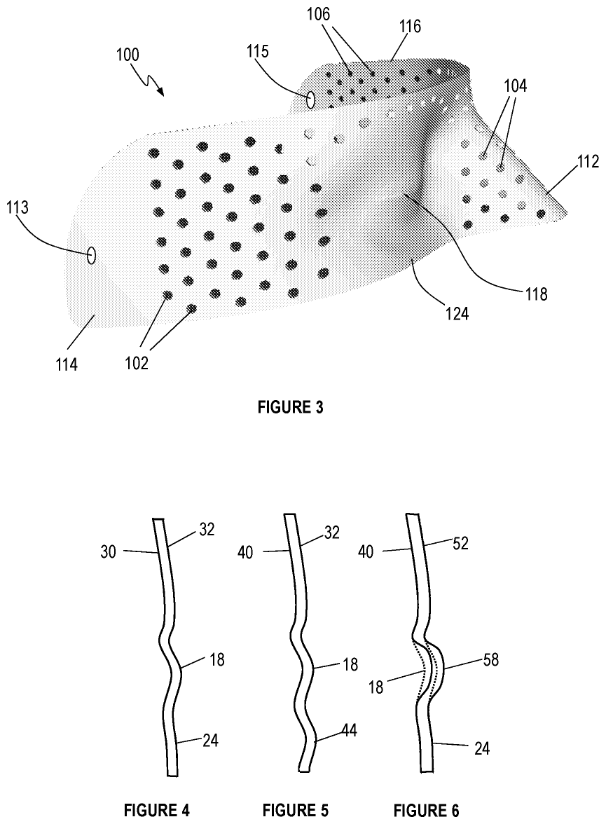

Turning now to FIG. 3, an alternative embodiment of an eye mask 100 is shown. As for the embodiment of FIGS. 1 and 2, the side portions 114, 116 in FIG. 3 are integral with the bridge portion 112 such that the mask 100 is formed as a single piece. It will be appreciated, though, that at least part of the side portions 114, 116 may instead be rigidly coupled to the bridge portion 112 by appropriate fastening means.

First side portion 114 includes an orbital region 118 that is adapted to cover a right eyeball of the user, and an infraorbital region 124 that is configured to apply pressure to a corresponding infraorbital region of the face when the eye mask 100 is attached to the face of the user. Second side portion 116 includes like orbital and infraorbital regions (for the user's left eye) that are not shown in FIG. 3. The infraorbital region of the second side portion 116 is configured to apply pressure to a corresponding infraorbital region of the face (also referred to herein as the eye bag region) when the eye mask 100 is attached to the face of the user.

The eye mask 100 may include at least one through-hole 102 (in this case, a plurality of through-holes 102, 104, 106) to assist with breathability and to thus increase the level of comfort for the wearer. The eye mask 100 has a bridge portion 112 from which first side portion 114 and second side portion 116 extend. First side portion 114 may include an aperture 113 near an extremity thereof, and second side portion 116 may include an aperture 115 near an extremity thereof. Apertures 113, 115 may be used to attach a retaining component, such as a band or strap (not shown), for securing the eye mask 100 to a face of a user. Alternatively, the band may be attached to side portions 114, 116 in other ways, such as by staples, adhesives, or heat-sealing.

The through-holes 102 improve breathability surrounding the eye areas and allows for oxygenation of the retina.

The through-holes 102 can have a diameter of about 0.5 mm to about 10 mm. In certain embodiments, the through-holes 102 have a diameter of about 0.5 mm to about 2 mm. The through-holes can extend to the extremities of the eye mask. In some embodiments, parts of the first side portion 114 and/or the second side portion 116 may be formed as a mesh or micromesh to achieve better breathability.

The through-holes can be arrayed. An array of through-holes 102 is formed in the first side portion 114. Similarly, an array of through-holes 106 is formed in the second side portion 116. An array of through-holes 104 is also formed in the bridge portion 112. Forming arrays of through-holes in each portion of the mask 100 both increases breathability and reduces the weight of the mask 100, thus further aiding user comfort. The spacing between the through-holes in the array can be regulated in order to maintain the sturdiness of the eye mask. It was found that the maintaining the spacing to be within a range can be further advantageous for forming an eye mask which is less prone to breakage. For example, the spacing can be from about 3 mm to about 15 mm, about 3 mm to about 12 mm, about 3 mm to about 10 mm, about 3 mm to about 8 mm, or about 3 mm to about 6 mm. It will be appreciated, though, that holes could be formed in just one of the mask portions 112, 114, 116, for example, or just in the side portions 114, 116, in particular in the orbital regions 118 for example.

Aeration holes 102, 104, 106 may be included in the mask 100 using FFF and SLS printers, simply by omitting printing material in the respective hole locations. Alternatively, the holes may be printed using a secondary soluble material (e.g., PVA, and the like) when printing using FFF printers, after which the soluble material is removed by washing in water.

In certain embodiments, the orbital regions 18, 20, 118, and/or 120 may be offset to improve user comfort. For example, as shown in FIG. 6, orbital region 18 may be adjusted such that it bulges outwardly, i.e. projects further away from the internal (face-contacting) surface 40 of the eye mask 10. In one example, the 3D facial scan data may be adjusted such that the orbital region 18 is offset by between about 5 mm and about 7 mm, and thus does not contact the orbital region of the user's face when the mask 10 is worn, thus allowing for freer eye movement during REM sleep and improving user comfort. An exemplary adjusted orbital region 58 is shown in FIG. 6, in which the original orbital region 18 is shown in dotted outline.

The side portions of the eye mask may be made of a flexible polymer material. This material can be of a lower rigidity or lower shore A hardness than that used in the bridge portion. For example, the flexible polymer material may be selected from the group consisting of thermoplastic polyurethane (TPU), polylactic acid (PLA), thermoplastic elastomer (TPE), plasticized copolyamide TPE (PCTPE), nylon, Acrylonitrile-Butadiene Styrene (ABS) and a combination of any two or more thereof. For example, Agilus30, a PolyJet Photopolymer with superior tear-resistance and is capable of withstanding repeated flexing and bending can be used.

The side portions 14, 16, 114, 116 may have a flexural strength of about 10 MPa to about 50 MPa. Alternatively, the material of the side portions 14, 16, 114, 116 may be characterised by shore hardness. Embodiments may have a shore A hardness in the range from around 10 A to 100 A.

For 3D printing using Fused Filament Fabrication (FFF) printers, flexible filaments (e.g., TPU, Recreus Filaflex, MatterHackers Soft PLA, NinjaTek SemiFlex, Polymakr Polyflex, FormFutura FlexiFil TPC, taulman3D PCTPE, and the like) may be used to print the mask. For 3D printing using Selective Laser Sintering (SLS) printers, powder of flexible materials (e.g., TPU, TPE, and the like) may be used.

3D printers such as Stratasys Objet500 Connex3 3D can be used for advanced 3D printing processes. Other non-limiting examples are Stratasys J750 liquid resin-based 3D printer for printing Vero+Tango material mix, and filament-based 3D printers such as BCN3D Sigma and Prusa i3 to print Nylon and TPU materials.

In some embodiments, as mentioned above, the bridge portion 12 or 112 may be formed from a material that is more rigid than the material of the side portions 14, 16 (or 114, 116). For example, the bridge portion 12 may be made of a material selected from the group consisting of polylactic acid (PLA), acrylonitrile butadiene styrene (ABS), nylon, and polycarbonate. The bridge portion 12 may have a flexural strength of about 50 MPa to about 100 MPa. Alternatively, the material of the bridge portion 12 may be characterised by its shore hardness. For example, the bridge portion 12 may have a shore A hardness ranging from about 50 A to 100 A.

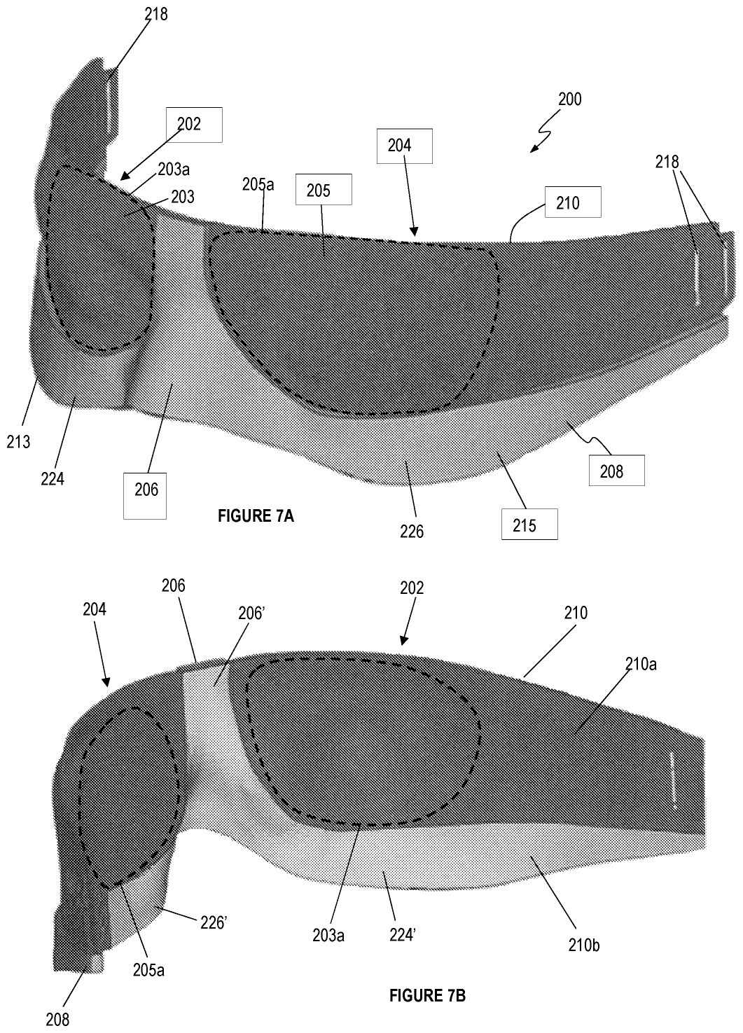

Turning now to FIGS. 7A and 7B, another embodiment 200 of an eye mask is shown. Side portions, generally indicated at 202 and 204, extend from a bridge portion 206. Side portion 202 includes an upper, flexible portion 203, and a lower, rigid, portion 213. Similarly, side portion 204 includes an upper, flexible, portion 205 and a lower, rigid, portion 215. The lower rigid portions 213 and 215 are part of a frame structure 208 which includes bridge portion 206 intermediate the lower rigid portions 213, 215.

The eye mask 200 has, at each of its sides, at least one elongate aperture 218 for receiving a retaining component, such as a strap, for securing the eye mask 200 to a user's head.

In the example of FIGS. 7A and 7B, the flexible upper portions 203 and 205 cover the orbital regions of the user when in use, while the rigid lower portions 213, 215 that are either side of bridge portion 206 include respective infraorbital regions 224, 226 that, being rigid, are configured to apply pressure to a corresponding infraorbital region of the face when the eye mask is attached to the face of the user. The orbital regions, which are generally indicated at 203a and 205a in FIGS. 7A and 7B, of the side portions 202, 204 are thus less rigid than the frame element 208.

As for the embodiments of FIGS. 1 to 3, the infraorbital regions 224, 226 may be outwardly recessed or indented so as to include one or more convex protrusions, such as the infraorbital region 44 with convex protrusion shown in FIG. 5. As for other embodiments disclosed herein, protrusions of varying depth at the infraorbital regions 224, 226 allow varying pressure to be applied to a corresponding infraorbital region of the face when the eye mask is attached to the face of the user.

In some embodiments, the upper portions 203, 205 are formed from a different material compared to the lower portions 213, 215 of frame element 208. The upper portions 203, 205 can be formed using a material with a lower rigidity and/or a lower shore A hardness than the frame element 208. Advantageously, this allows greater comfort to the user as, for example, it provides greater freedom of eye movement during, for example, REM sleep. The higher rigidity of the bridge portion 206 of frame element 208 allows the eye mask 200 to be held in position during sleep by, for example, resting on the dorsum nasi area of the nasal region of the user.

The upper portions 203, 205 can be formed with a thinner cross-section compared to the bridge portion 206. The upper portions 203, 205 can, for example, have a thickness of about 0.2 mm to about 5 mm, about 0.2 mm to about 4 mm, about 0.2 mm to about 2 mm or about 0.4 mm to about 1 mm. This is further advantageous as it reduces the pressure on the eye of the subject, and accordingly provides greater comfort to the user. Additionally, the thinner, more flexible material also prevents or substantially reduces skin indentation along the edges of the eye mask 200.

In combination with the bridge portion 206, the lower portions 213, 215 provide the eye mask with a structural frame ‘exoskeleton’, which provides balance, limits movement at the nose bridge, and increases compression under the eye. Additionally, as the upper portions 203, 205 can be made using a less rigid (and possibly softer material), the perceived comfort by the user is expected be higher. This is due to the greater flexibility around the eyeballs while maintaining under and upper eye area compression.

In some embodiments, the orbital regions 203a, 205a are carried on a liner attached to the frame element. For example, a liner 210 may be attached to frame element 208. As shown in the rear view of FIG. 7B, in which changes in shading indicate regions of different rigidity, the liner 210 may have a first part 210a and a second part 210b, where the first part 210a carries the orbital regions 203a, 205a, and the second part 210b is attached to the frame element 208. The second part 210b of the liner 210 is in contact with the infraorbital regions 224, 226 of the eye-contacting portions 202 and 204. Corresponding parts 224′ and 226′ of the liner in the second part 210b are aligned with the infraorbital regions 224 and 226. Likewise, a portion of the liner 206′ is aligned with the bridge portion 206.

The infraorbital regions 224, 226 can extend substantially to an extremity of the eye mask. The extension of the infraorbital region 224 or 226 can extend to a position near the apertures 218. While the infraorbital region is shown to extend to a position below the aperture 218 in FIG. 7A, the extension can alternatively be above the aperture 218 or be both above and below the aperture 218. Advantageously, this provides additional strength to the eye mask such that it breaks or tears less readily. Additionally, the extension of the infraorbital region 224, 226 can loop around the aperture 218. This reinforces the aperture 218 such that it will not tear when subjected to a tension by the retaining component securing the eye mask 200 to the user's head.

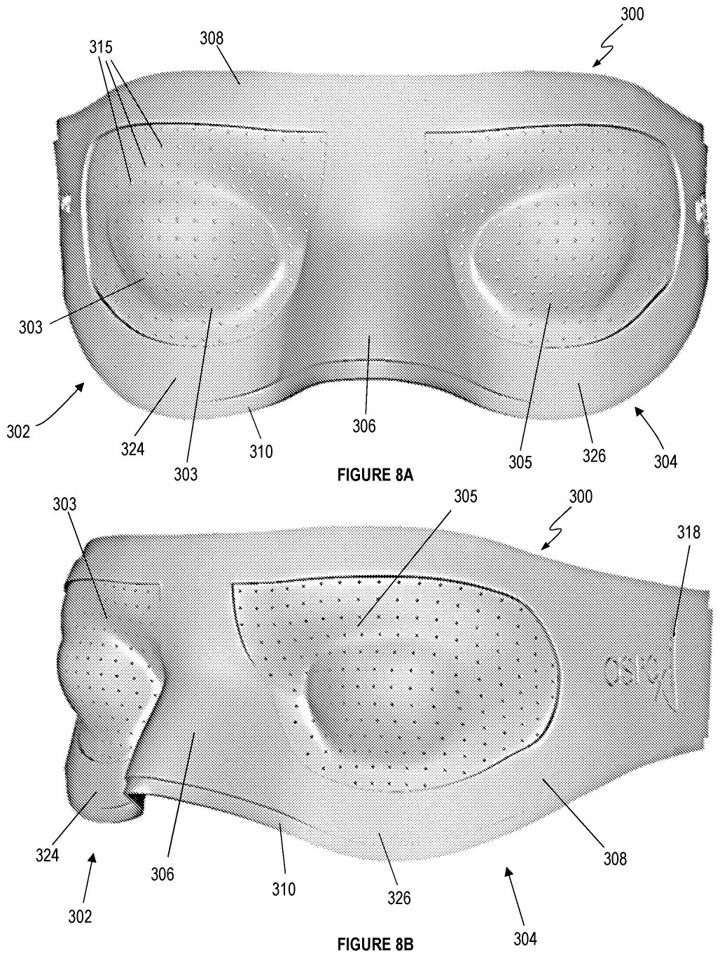

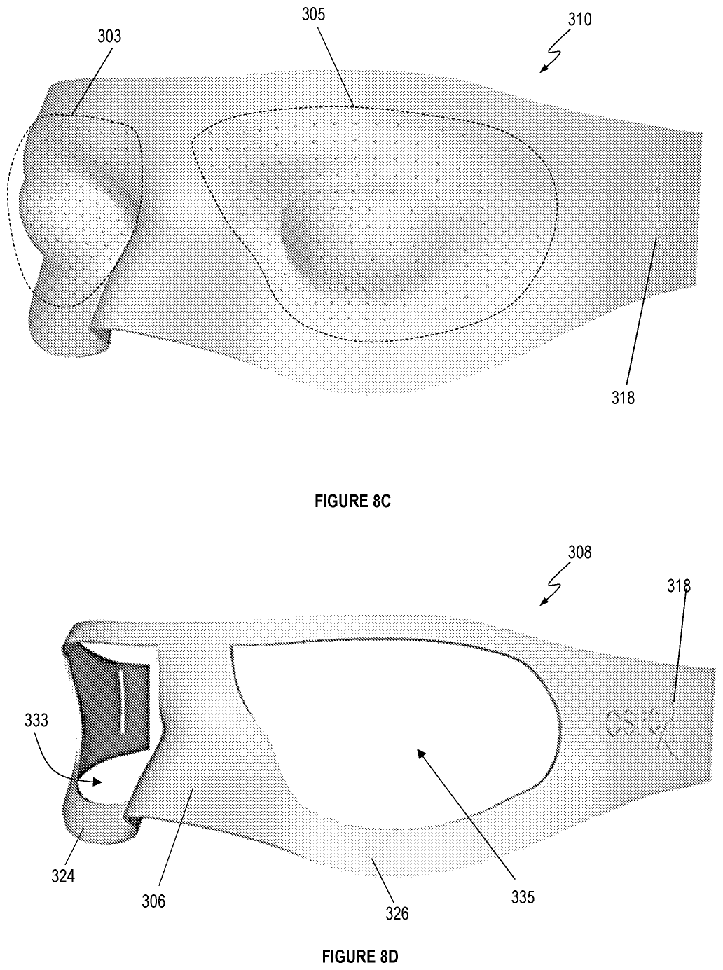

FIGS. 8A-8D show another embodiment of an eye mask. The eye mask 300 can comprise an inner lining 310 and an outer frame 308. The inner lining 310 is assembled together with the outer frame 308 to form the eye mask 300. The eye mask 300 has a first side portion 302 and a second side portion 304, each of which extends from a bridge portion 306. The frame 308 has, at its lower part, a first infraorbital region 324 associated with the first side portion 302, and a second infraorbital region 326 associated with the second side portion 304. The first and second infraorbital regions 324, 326 are configured to apply pressure to a corresponding infraorbital region of the face when the eye mask 300 is attached to the face of the user. For example, this may be by way of convex protrusions on the opposite (user-facing) surface of the mask 300 in the infraorbital regions 324, 326. The eye mask 300 also has orbital regions 303, 305 above the infraorbital regions 324, 326.

The inner lining may be made of a flexible polymer material as discussed above in relation to other embodiments. The inner lining may also be a smooth and soft material. The inner lining can also be a non porous and non leaching material, which is advantageous for functionalisation with cosmetic and/or active ingredients.

The eye mask 300 further includes apertures 318 for attachment of a retaining component (not shown) for securing the eye mask 300 to the user's face.

The inner lining 310 can be made from a less rigid (or softer) material than the outer frame 308 and substantially covers the orbital region, infraorbital region and nasal region of the user's face. The outer frame 308 can be made from a more rigid (or harder) material and substantially covers the nasal region of the user's face. The outer frame 308 can also extend to the extremities of the eye mask 300, such that it forms a frame (or perimeter) around at least the orbital regions 303 and 305. For example, as shown in FIGS. 8C and 8D, the frame 308 may have cut-out portions 333 and 335 such that when the liner 310 is combined with the frame 308, orbital regions 303 and 305 (respectively) remain exposed.

In some embodiments, inner lining 310 may have a plurality of apertures 315 to improve breathability of the mask 300. For example, the apertures 315 may be provided in an array, and may be provided at least in the orbital regions 303 and 305.

The inner lining 310 can be combined with the outer frame 308 to form the eye mask 300. To this end, the bridge portion 306 comprises a layered structure of portions of the inner lining 310 and outer frame 308. The orbital regions 303, 305 of the side portions 302, 304 are carried on the inner lining 310, while the infraorbital regions 324, 326 of the side portions 302, 304 comprise a layered structure of portions of inner lining 310 and outer frame 308. In use, the inner lining 310 is positioned in contact with the user's face. This is advantageous as the inner lining (a softer, smoother material) provides for comfort for overnight use.

The layered structure of inner lining 310 and outer frame 308 at the infraorbital regions 324, 326 of the eye mask 300, and the inner lining 310 only at the orbital regions 303, 305 of the eye mask 300 is advantageous as the inner lining 310 can be made to be smooth (and breathable, if apertures 315 are included) for greater comfort while the harder exoskeleton 308 provides support, structural balance and compression on the infraorbital regions 326, 326.

In addition to being made from a material with a lower rigidity, the inner lining 310 can have a thickness of about 0.1 mm to about 10 mm, or about 0.2 mm to about 8 mm, about 0.2 mm to about 6 mm, about 0.2 mm to about 4 mm, or about 0.2 mm to about 2 mm. In some embodiments, the inner lining 310 has a thickness of about 0.5 mm.

The outer frame 308 can have a thickness of about 1 mm to about 10 mm, about 1 mm to about 8 mm, about 1 mm to about 6 mm, about 1 mm to about 4 mm, about 1 mm to about 2 mm. In some embodiments, the outer frame 308 has a thickness of about 1 mm.

The inner lining 310 can extend beyond the boundaries of the outer frame 308, as shown in FIGS. 8A and 8B. The inner lining 310 can, for example, be extended about 0.2 mm to about 10 mm, about 0.5 mm to about 8 mm, about 0.5 mm to about 5 mm, or about 1 mm to about 5 mm. This can help to minimize skin indentations when the user wears the eye mask 300 overnight.

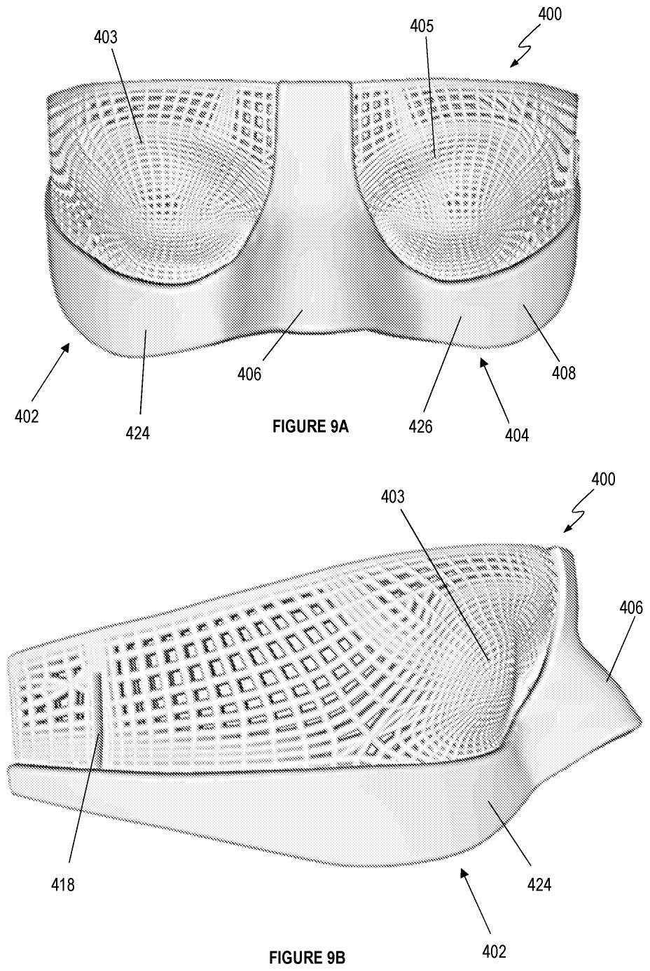

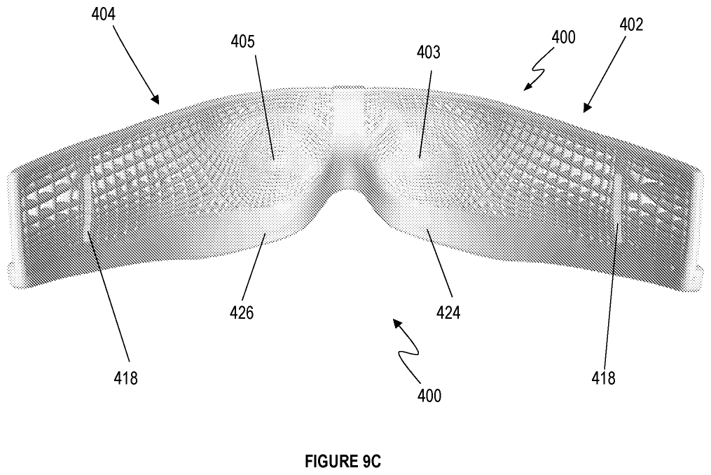

FIGS. 9A to 9C show another embodiment of an eye mask 400. In this embodiment, a rigid lower portion 408 spans across a bridge portion 406 and side portions 402, 404. The bridge portion 406 and lower portions of the side portions 402, 404 are both thickened to reinforce the compression effect. An upper portion of the eye mask 400 includes a mesh that covers the orbital regions 403, 405 of the side portions 402, 404 to improve breathability and at the same time provides rigidity for making the eye mask 400 more durable.

Similar with other embodiments disclosed herein, the orbital regions 403, 405 of the side portions 402, 404 (meshed area) and the bridge portion 406 (thickened area) can be printed with different materials and/or with different hardness.

An inner lining (not shown) can be further layered on the internal (user-facing) surface of the eye-mask 400. In addition to increasing the comfort level for the user, the inner lining provides a surface for application of cosmetics and/or active ingredients which can aid in the reduction of periorbital puffiness.

In certain embodiments, an additional treatment component may be applied to the internal surface 30, 40 of the eye mask 10, in the infraorbital regions (24, 26, 44) of the eye mask 10 (and likewise for the other eye masks 100, 200, 300, 400 disclosed herein). For example, an active agent in the form of or impregnated in a coating or liner may be applied in the infraorbital regions. Example active agents are a moisturiser, a coolant, an anti-aging agent, and an anti-oxidant. Combinations of any two or more of these may also be used.

An eye mask 10, 100, 200, 300, 400 may be included as part of a kit for treating periorbital puffiness. For example, a plurality of such eye masks may be provided, respective eye masks having respective infraorbital regions, the respective infraorbital regions of respective eye masks being configured to apply progressively greater pressure to the corresponding infraorbital regions of the face. In one example, the eye masks in the kit have infraorbital regions having protrusions of progressively shallower depth.

Certain embodiments relate to a method of manufacturing an eye mask for treating and/or preventing periorbital puffiness in a user. Example embodiments are discussed with reference to the eye mask 10, but it will be appreciated that the method may also be applied to manufacture any other embodiment including the eye masks 100, 200, 300 or 400.

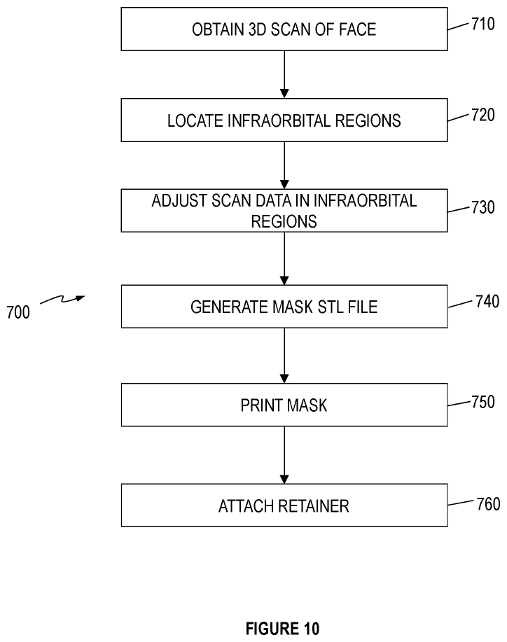

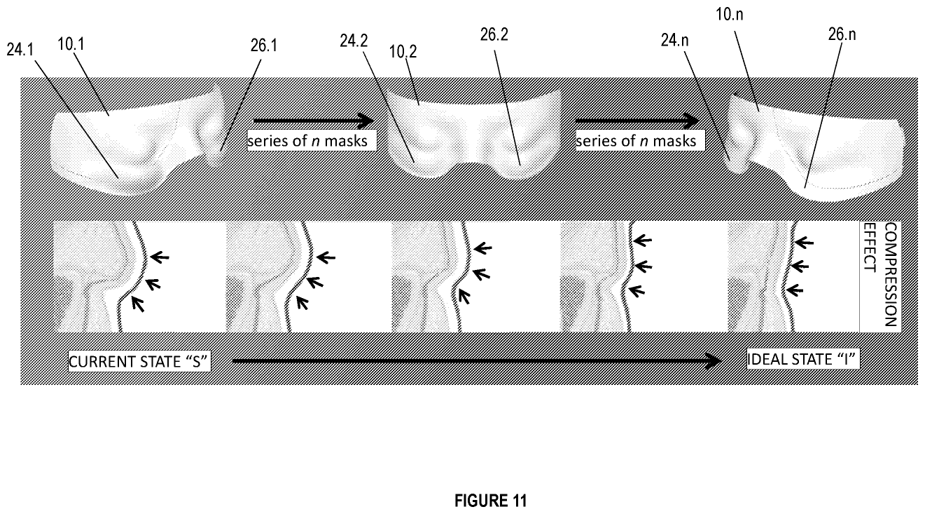

For example, as shown in FIG. 10, a method 700 may include a step 710 of obtaining three-dimensional (3D) facial scan data of the user. The scan can be generated by an off-the-shelf device such as a Face Camera Pro of Bellus 3D, Inc. Other types of facial scanners, such as hand-held scanners, may also be used.

Next, the method 700 may include a step 720 of analysing the facial scan data to determine a boundary of the orbicularis ocularis muscle. The analysis step 720 may also include determining a boundary of an eye bag area (infraorbital region) on the face of the user.

Optionally, the method 700 may include a step 730 of adjusting the 3D facial scan data in the infraorbital regions, as will be explained in more detail below. Once the 3D facial scan data are obtained (and optionally, adjusted), the method proceeds to step 740, where a mask design (for example, in 3D-printable format such as an STL file) is generated. The mask design includes two side portions extending from a bridge portion. Each side portion includes an orbital region, and an infraorbital region that is configured to apply pressure to a corresponding infraorbital region of the face of the user when the eye mask is attached to the face. Exemplary mask designs are described above and depicted in FIGS. 1-9.

Next, at step 750, a mask body is fabricated according to the mask design, for example by 3D printing. In certain embodiments, the mask body is fabricated by fused filament fabrication, selective laser sintering, or stereolithography. In other embodiments, the eye mask is fabricated in two parts. The inner lining and the outer frame can be fabricated separately and combined using an appropriate adhesive or bonding method. For example, these two components can be heat bonded. Advantageously, this allows the two components to be made from different materials. For example, the inner lining can be printed from a thermoplastic filament with a lower melting point such that a smooth finish can be directly obtained for improved comfort and hygiene. In contrast, as the outer frame is not in contact with the user's face, a fine finish is not required and a crude 3D printed product can be further processed by, for example, sanding.

Finally, at step 760, a retaining component is provided at the mask body for attaching the eye mask to the face of the user. For example, a retainer such as a strap or belt may be attached to the mask body, such as by stapling, gluing, heat sealing, or threading through holes in the side portions of the mask body (for example, as shown in FIGS. 1 and 3).

As mentioned above, the 3D facial scan data may be modified prior to generating the mask design. This may be done in a number of ways.

For example, the 3D facial scan data may be modified to include one or more convex protrusions (facing outwardly from the user) in each infraorbital region of the eye mask.

In one example, where a mask or set of masks is to be used for treating an existing eye bag, a new 3D facial scan data set can be then created to represent the preferred eye contour without the existing eye bag i.e. the individual facial profile without a convex shape caused by the excess fluid. This data set represents the desired end shape and profile (mask data Z=preferred facial contour).

In some embodiments, the method 700 may be used to produce a set of masks for treating periorbital puffiness. For example, the 3D facial scan data can be incrementally changed to create mask designs having progressively lower amounts of indentation at the infraorbital regions 24, 26, with each respective mask design being used to print a respective mask. The set of mask designs may slowly and incrementally move from the original facial scan data to a final data set Z representing the preferred facial contour. For example, for small eye bags, 4 mask sets might be created (mask data sets A, B, C, Z). For larger, more pronounced eye bags, more masks may be required so that the compression effect is kept within reasonable limits to allow time for the skin to adjust. In this case 10 mask profile sets might be created to get from the original data set to mask data set Z.

In another example, a convex shape of the existing eye bags may be determined from the facial scan data, and then inverted and transformed into a matching shape using CAD, thus producing a mask design that will result in a compression effect when the subject wears the mask. To modify the compression effect gradually over time, an adjustable strap (such as a hook-and-loop belt) may be used to retain the mask on the user's head, with the user being able to adjust the tightness of the adjustable strap over time to gradually increase the compression effect and push the infraorbital region towards the desired facial contour.

In another example, the 3D facial scan data may be modified such that the bridge portion of the mask design is thicker than the side portions.

In a further example, the 3D facial scan data may be modified to include one or more apertures in the mask design, such that when the mask design is printed, one or more through-holes are formed in the eye mask. An array of such holes may be included in the mask design.

In some embodiments, an active agent may be applied to the infraorbital region of the eye mask, either before or after attaching the retainer to the mask body. For example, the active agent may be included in a coating or a liner. The active agent may be selected from the group consisting of a moisturiser, a coolant, anti-aging agent, anti-oxidant and a combination thereof.

Embodiments also relate to a method of treating periorbital puffiness in a subject in need thereof, comprising administering an eye mask as disclosed herein. For example, in some embodiments, the method may be used to treat patients suffering from periorbital puffiness following eye or other facial surgery. To this end, a single eye mask, or a kit of eye masks each configured to provide different amounts of infraorbital compression, as disclosed herein, may be provided as part of a treatment regime.

In one example of a treatment regime for a patient post-surgery, a scan of the patient's face is obtained pre-surgery. The method 700 of FIG. 7 may then be performed post-surgery to generate a series of masks, with the scan data in the infraorbital regions being incrementally adjusted from the initial infraorbital shape towards the original infraorbital shape obtained from the pre-surgery scan. A first mask is attached after the surgery and kept in place for 3-14 days to prevent or minimise swelling throughout the day and night. In some embodiments the mask data may be modified to allow for and accommodate the thickness of bandages, gauze, stitches etc in particular areas, while still maintaining the desired compression effect.

Embodiments further relate to an eye mask as disclosed herein for use in the treatment of periorbital puffiness.

Periorbital puffiness or periorbital edema is a term for swelling around the eyes. Periorbital edema can occur in just one eye or both at the same time. One cause of periorbital edema is inflammation that causes fluid build up around the eye.

Some common causes for periorbital edema include mononucleosis (viral eye infection), irregular sleep, high-salt diet, high alcohol consumption, smoking, allergies, skin disorders, aging, crying, thyroid disorders, periorbital cellulitis (skin condition caused by infection and inflammation of the eyelid and the skin around the eyes), Chagas disease, nephrotic syndrome, trichinosis, dysfunctional tear glands, obstruction of the superior vena cava, conjunctivitis, and trauma to the eye. Periorbital puffiness can also result from eyelid surgery, chemical burns, eyelid sunburn, sinusitis, eyelid skin infection such as impetigo and erysipelas, blepharitis, stye, chalazion, ocular herpes, and Graves' disease.

A description of some exemplary embodiments of the present disclosure is contained in one or more of the following numbered statements:

Statement 1. An eye mask for treating and/or preventing periorbital puffiness, including:
two side portions extending either side of a bridge portion; and
a retaining component for attaching the eye mask to a face of a user;
wherein each side portion includes an orbital region adapted to cover an eyeball of the user, and an infraorbital region that is configured to apply pressure to a corresponding infraorbital region of the face when the eye mask is attached to the face of the user.

Statement 2. The eye mask according to statement 1, wherein each infraorbital region of the eye mask includes one or more protrusions that extend away from a user-facing surface of the eye mask.

Statement 3. The eye mask according to statement 1 or statement 2, wherein each infraorbital region of the eye mask includes an elongate compression region.

Statement 4. The eye mask according to statement 3, wherein the elongate compression regions are crescent-shaped.

Statement 5. The eye mask according to any one of statements 2 to 4, wherein the one or more protrusions each have a depth in the range between about 1 mm and about 10 mm.

Statement 6. The eye mask according to any one of statements 1-5, including a frame element that includes the bridge portion and the infraorbital regions of the side portions.

Statement 7. The eye mask according to statement 6, wherein the orbital regions of the side portions are less rigid than the frame element.

Statement 8. The eye mask according to statement 6 or statement 7, wherein the orbital regions are carried on a liner attached to the frame element.

Statement 9. The eye mask according to statement 8, wherein the liner extends at least partly beyond an edge of the frame element.

Statement 10. The eye mask according to any one of statements 1-9, wherein the side portions are made of a flexible polymer material.

Statement 11. The eye mask according to statement 10, wherein the flexible polymer material is selected from the group consisting of thermoplastic polyurethane (TPU), polylactic acid (PLA), thermoplastic elastomer (TPE), plasticized copolyamide TPE (PCTPE), nylon, and a combination of any two or more thereof.

Statement 12. The eye mask according to any one of statements 1 to 11, wherein the side portions have a shore A hardness in the range from about 10 A to 100 A.

Statement 13. The eye mask according to any one of statements 1 to 12, wherein the side portions are rigidly coupled to, or are integral with, the bridge portion.

Statement 14. The eye mask according to any one of statements 1 to 13, wherein the bridge portion is more rigid than the side portions.

Statement 15. The eye mask according to statement 14, wherein the bridge portion is made of a material selected from the group consisting of polylactic acid (PLA), acrylonitrile butadiene styrene (ABS), nylon, and polycarbonate.

Statement 16. The eye mask according to statement 14 or statement 15, wherein the bridge portion has a shore A hardness in the range from about 50 A to about 100 A.

Statement 17. The eye mask according to statement 14, wherein the bridge portion is made of the same material as the side portions, and has a greater thickness than the side portions.

Statement 18. The eye mask according to any of statements 1 to 17, including at least one through-hole.

Statement 19. The eye mask according to statement 18, including an array of through-holes.

Statement 20. The eye mask according to statement 19, including an array of through-holes in each orbital region.

Statement 21. The eye mask according to any of statements 1 to 20, wherein the retaining component includes a strap.

Statement 22. The eye mask according to statement 21, wherein the strap is adjustable to vary the pressure applied by the infraorbital regions of the eye mask.

Statement 23. The eye mask according to any one of statements 1 to 22, including an active agent applied to the infraorbital regions of the eye mask, the active agent being selected from the group consisting of a moisturiser, a coolant, an anti-aging agent, an anti-oxidant and a combination of any two thereof.

Statement 24. A kit for treating periorbital puffiness, including a plurality of eye masks according to any one of statements 1 to 23, respective eye masks having respective infraorbital regions, the respective infraorbital regions of respective eye masks being configured to apply progressively greater pressure to the corresponding infraorbital regions of the face.

Statement 25. A method of manufacturing an eye mask for treating and/or preventing periorbital puffiness in a user, including:
obtaining three-dimensional (3D) facial scan data of the user;
generating, from the 3D facial scan data, a mask design, the mask design including two side portions extending either side of a bridge portion;
wherein each side portion includes an orbital region adapted to cover an eyeball of the user, and an infraorbital region that is configured to apply pressure to a corresponding infraorbital region of the face of the user when the eye mask is attached to the face;
fabricating a mask body according to the mask design; and
providing, at the mask body, a retaining component for attaching the eye mask to the face of the user.

Statement 26. The method according to statement 25, wherein the mask body is fabricated by 3D printing.

Statement 27. The method according to statement 26, wherein the mask body is fabricated by fused filament fabrication, selective laser sintering, or stereolithography.

Statement 28. The method according to any one of statements 25 to 27, including modifying the 3D facial scan data to include one or more protrusions in each infraorbital region of the eye mask.

Statement 29. The method according to statement 28, wherein the one or more protrusions each have a depth in the range between about 1 mm and about 10 mm.

Statement 30. The method according to any one of statements 25 to 29, wherein the bridge component is more rigid than the side portions.

Statement 31. The method according to statement 30, wherein the bridge portion is formed with a greater thickness than the side portions.

Statement 32. The method according to any of statements 25 to 31, including forming at least one through-hole in the eye mask.

Statement 33. The method according to statement 32, including forming an array of through-holes in the eye mask.

Statement 34. The method according to any one of statements 25 to 33, including applying an active agent to the infraorbital region of the eye mask, the active agent being selected from the group consisting of a moisturiser, a coolant, an anti-aging agent, an anti-oxidant and a combination of any two thereof.

Statement 35. An eye mask according to any one of statements 1-23, or a kit according to statement 24, for use in the treatment of periorbital puffiness.

Statement 36. A method of treating periorbital puffiness in a subject in need thereof, comprising administering one or more eye masks according to any one of statements 1-23.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. An eye mask for treating and/or preventing periorbital puffiness, including:
two side portions extending either side of a bridge portion; and
a retaining component for attaching the eye mask to a face of a user;
wherein each side portion includes an orbital region adapted to cover an eyeball of the user, and an infraorbital region that is configured to apply pressure to a corresponding infraorbital region of the face when the eye mask is attached to the face of the user.

2. The eye mask according to claim 1, wherein each infraorbital region of the eye mask includes one or more protrusions that extend away from a user-facing surface of the eye mask.

3. The eye mask according to claim 1, wherein each infraorbital region of the eye mask includes an elongate compression region.

4. The eye mask according to claim 1, wherein each infraorbital region of the eye mask includes an elongate compression region, and wherein the elongate compression regions are crescent-shaped.

5. The eye mask according to claim 1, including a frame element that includes the bridge portion and the infraorbital regions of the side portions.

6. The eye mask according to claim 5, wherein the orbital regions of the side portions are less rigid than the frame element.

7. The eye mask according to claim 5, wherein the orbital regions are carried on a liner attached to the frame element.

8. The eye mask according to claim 5, wherein the orbital regions are carried on a liner attached to the frame element, and wherein the liner extends at least partly beyond an edge of the frame element.

9. The eye mask according to claim 1, wherein the side portions are made of a flexible polymer material.

10. The eye mask according to claim 1, wherein the side portions are made of a flexible polymer material, the flexible polymer material is selected from the group consisting of thermoplastic polyurethane (TPU), polylactic acid (PLA), thermoplastic elastomer (TPE), plasticized copolyamide TPE (PCTPE), nylon, and a combination of any two or more thereof.

11. The eye mask according to claim 1, wherein the side portions have a shore A hardness in the range from about 10 A to 100 A.

12. The eye mask according to claim 1, wherein the bridge portion is more rigid than the side portions.

13. The eye mask according to claim 1, wherein the bridge portion is made of a material selected from the group consisting of polylactic acid (PLA), acrylonitrile butadiene styrene (ABS), nylon, and polycarbonate.

14. The eye mask according to claim 1, wherein the bridge portion is made of the same material as the side portions, and has a greater thickness than the side portions.

15. The eye mask according to claim 1, including an array of through-holes.

16. The eye mask according to claim 1, including an array of through-holes in each orbital region.

17. The eye mask according to claim 1, including an active agent applied to the infraorbital regions of the eye mask, the active agent being selected from the group consisting of a moisturiser, a coolant, an anti-aging agent, an anti-oxidant and a combination of any two thereof.

18. A kit for treating periorbital puffiness, including a plurality of eye masks according to claim 1, respective eye masks having respective infraorbital regions, the respective infraorbital regions of respective eye masks being configured to apply progressively greater pressure to the corresponding infraorbital regions of the face.

19. A method of manufacturing an eye mask for treating and/or preventing periorbital puffiness in a user, including:
obtaining three-dimensional (3D) facial scan data of the user;
generating, from the 3D facial scan data, a mask design, the mask design being in accordance with claim 1;
fabricating a mask body according to the mask design; and
providing, at the mask body, a retaining component for attaching the eye mask to the face of the user.

20. The method according to claim 19, wherein the mask body is fabricated by 3D printing, such as by fused filament fabrication, selective laser sintering, or stereolithography.
